(19) United States
(12) Patent Application Publication
(10) Pub. No.: US 2023/0033661 A1
(43) Pub. Date: Feb. 2, 2023

(54) MOBILE MEDICAL TREATMENT SERVICE SYSTEM AND MOBILE MEDICAL TREATMENT SERVICE METHOD

(71) Applicants: HYUNDAI MOTOR COMPANY, Seoul (KR); Kia Corporation, Seoul (KR)

(72) Inventor: Keunwoong KIM, Incheon (KR)

(73) Assignees: HYUNDAI MOTOR COMPANY, Seoul (KR); Kia Corporation, Seoul (KR)

(21) Appl. No.: 17/869,353

(22) Filed: Jul. 20, 2022

(30) Foreign Application Priority Data
Jul. 26, 2021 (KR) 10-2021-0097887

(51) Int. Cl. G16H 40/20; G16H 10/60

(52) U.S. Cl. CPC G16H 40/20; G16H 10/60; G06Q 10/1095

(57) ABSTRACT

A mobile medical treatment service system, including: a data system that receives a plurality of mobile medical treatment appointment requests and a plurality of pieces of medical treatment appointment information from a plurality of user terminals, and that stores the plurality of pieces of medical treatment appointment information; and a central management device that derives the plurality of pieces of medical treatment appointment information from the data system, that classifies the plurality of mobile medical treatment appointments into a plurality of groups based on a desired medical treatment date among the derived medical treatment appointment information, and determines the medical treatment schedule according to the number of appointments belonging to the plurality of groups, that calculates a medical treatment target area based on residential areas of the plurality of users among the derived medical treatment appointment information, and that determines a medical treatment position within the medical treatment target area.

### CROSS-REFERENCE TO RELATED APPLICATION

This application claims priority to and the benefit of Korean Patent Application No. 10-2021-0097887 filed in the Korean Intellectual Property Office on Jul. 26, 2021, the entire contents of which are incorporated herein by reference.

### TECHNICAL FIELD

The present disclosure relates to a mobile medical treatment service system and a mobile medical treatment service method.

### BACKGROUND

A current medical service is a medical institution-centered service, and thus needs to be improved to a patient-centered medical service. In order to realize this, a mobile medical treatment service system that can diagnose and treat in areas in which no medical institutions are located is required.

In addition, when a contagious disease such as coronavirus disease occurs, patients may not be able to visit medical institutions due to possibility of disease infection. In this case, a mobile medical treatment service system that can move to the area in which the patient is located is required.

The above information disclosed in this Background section is only for enhancement of understanding of the background of the disclosure, and therefore it may contain information that does not form the prior art that is already known in this country to a person of ordinary skill in the art.

### SUMMARY

The present disclosure has been made in an effort to provide a mobile medical treatment service system and a mobile medical treatment service method.

An embodiment of the present disclosure provides a mobile medical treatment service system, including: a data system that receives a plurality of mobile medical treatment appointment requests and a plurality of pieces of medical treatment appointment information from a plurality of user terminals, and that stores the plurality of pieces of medical treatment appointment information; and a central management device that derives the plurality of pieces of medical treatment appointment information from the data system, that classifies a plurality of mobile medical treatment appointments into a plurality of groups based on a desired medical treatment date among the derived plurality of pieces of medical treatment appointment information, and determining a medical treatment schedule according to a number of appointments belonging to the a plurality of groups, that calculates a medical treatment target area based on residential areas of a plurality of users among the derived plurality of pieces of medical treatment appointment information, and that determines a medical treatment position within the medical treatment target area by machine-learning a relationship between an accumulated medical treatment position and a participation rate. The participation rate may be a ratio of users that have actually received a mobile medical treatment service among the plurality of users.

The central management device may include: a data augmentation part that reads identification information, a desired medical treatment date, a residential area, and a symptom from each of the pieces of plurality of medical treatment appointment information from the data system; a classification part that generates a plurality of medical treatment appointment groups by classifying the plurality of mobile medical treatment appointments according to the desired medical treatment date, the residential area, and the symptom of each of the read plurality of pieces of medical treatment appointment information; and a medical treatment plan determining part that counts a number of the mobile medical treatment appointments belonging to each of the plurality of medical treatment appointment groups, selects a medical treatment appointment group with the highest number of the counted mobile medical treatment appointments among the plurality of medical treatment appointment groups as a medical treatment scheduled group, and determines a medical treatment plan based on a desired medical treatment date, a symptom, and a residential area of the medical treatment scheduled group.

The medical treatment plan determining part may determine a medical treatment schedule based on the desired medical treatment date of the medical treatment scheduled group, determine the symptom of the medical treatment scheduled group as a treatment subject, and determine the residential area of the medical treatment scheduled group as the medical treatment target area.

The medical treatment plan determining part may learn the relationship between the accumulated medical treatment position and the participation rate through deep learning, and may determine a specific position within the medical treatment target area as the medical treatment position.

The central management device may include a medical treatment appointment supplement part that derives a medical treatment appointment group that does not match the medical treatment plan but has the same symptom among the plurality of medical treatment appointment groups, derive a phone number from personal information of a medical treatment appointment changing user who is a mobile medical treatment service appointment user belonging to the derived medical treatment appointment group, and transmit the medical treatment plan to the phone number of the medical treatment appointment changing user.

The medical treatment appointment supplement part may derive a phone number from personal information of a preliminary service user stored in the data system to transmit the medical treatment plan to the phone number of the preliminary service user, and the preliminary service user may be an existing service user using a mobile medical treatment service with the same symptom as the medical treatment plan.

The central management device may further include a medical treatment appointment supplement part that derives a medical treatment appointment group that does not match the medical treatment plan but has the same symptom among the plurality of medical treatment appointment groups, and derive an identification number of a medical treatment appointment changing user who is a mobile medical treatment service appointment user belonging to the derived medical treatment appointment group to transmit it to the data system.

The data system may transmit the medical treatment plan to a phone number corresponding to the derived identification number, receive a changed desired medical treatment date from a user terminal of the medical treatment appointment changing user, and transmit the desired medical treatment date and identification information of the medical treatment appointment changing user to the central management device.

The data system may include an appointment server that stores at least one of the plurality of pieces of medical treatment appointment information in response to at least one of the plurality of mobile medical treatment appointment requests for the mobile medical treatment service and additionally processes an appointment schedule change in response to a request from the central management device.

The appointment server may include: an appointment database that stores personal information, a desired treatment date, a residential area, and a symptom for each mobile medical treatment service appointment user; and a medical treatment record database that stores medical treatment information about a service user who has previously used a mobile medical treatment service.

Another embodiment of the present disclosure provides a mobile medical treatment service method, including: receiving, by a data system, a plurality of mobile medical treatment appointment requests and a plurality of pieces of medical treatment appointment information from a plurality of user terminals, and storing the plurality of pieces of medical treatment appointment information; deriving, by a central management device, a plurality of pieces of medical treatment appointment information from the data system; classifying, by the central management device, a plurality of mobile medical treatment appointments into a plurality of groups based on a desired medical treatment date among the derived plurality of pieces of medical treatment appointment information; determining, by the central management device, a medical treatment schedule according to a number of appointments belonging to the plurality of groups; calculating, by the central management device, a medical treatment target area based on residential areas of a plurality of users among the derived plurality of pieces of medical treatment appointment information; learning, by the central management device, a relationship between an accumulated medical treatment position and a participation rate through deep learning; and determining, by the central management device, a medical treatment position in the medical treatment target area based on a learned result. The participation rate may be a ratio of users that have actually received a mobile medical treatment service among the plurality of users.

The classifying of the plurality of mobile medical treatment appointments into the plurality of groups may include: reading, by the central management device, identification information, a desired medical treatment date, a residential area, and a symptom from each of the plurality of pieces of medical treatment appointment information from the data system; and generating, by the central management device, a plurality of medical treatment appointment groups by classifying the plurality of mobile medical treatment appointments in consideration of the desired medical treatment date, the residential area, and the symptom of each of the read plurality of pieces of medical treatment appointment information.

The determining may include: counting, by the central management device, the number of the mobile medical treatment appointments belonging to each of the plurality of medical treatment appointment groups; selecting, by the central management device, a medical treatment appointment group having the highest number of the mobile medical treatment appointments counted among the plurality of medical treatment appointment groups as a medical treatment scheduled group; determining, by the central management device, the medical treatment schedule based on a desired medical treatment date of the medical treatment scheduled group; determining, by the central management device, a symptom of the medical treatment scheduled group as a medical treatment subject; and determining, by the central management device, a residential area of the medical treatment scheduled group as the medical treatment target area.

The determining of the medical treatment position may include determining, by the central management device, a specific position in the medical treatment target area as the medical treatment position based on the learned result.

The mobile medical treatment service method may further include: deriving, by the central management device, a medical treatment appointment group that does not match a medical treatment plan but has the same symptom among the plurality of medical treatment appointment groups; deriving, by the central management device, a phone number from personal information of a medical treatment appointment changing user who is a mobile medical treatment service appointment user belonging to the derived medical treatment appointment group; and transmitting, by the central management device, the medical treatment plan to the phone number of the medical treatment appointment changing user.

The mobile medical treatment service method may further include: deriving, by the central management device, a phone number from personal information of a preliminary service user stored in the data system; and transmitting, by the central management device, the medical treatment plan to the phone number of the preliminary service user, wherein the preliminary service user is an existing service user using a mobile medical treatment service with the same symptom as the medical treatment plan.

The mobile medical treatment service method may further include: deriving, by the central management device, a medical treatment appointment group that does not match a medical treatment plan but has the same symptom among the plurality of medical treatment appointment groups; deriving, by the central management device, an identification number of a medical treatment appointment changing user who is a mobile medical treatment service appointment user belonging to the derived medical treatment appointment group; and transmitting, by the central management device, the derived identification number to the data system.

The mobile medical treatment service method may further include: transmitting, by the data system, the medical treatment plan to a phone number corresponding to the derived identification number; receiving, by the data system, a changed desired medical treatment date from a user terminal of the medical treatment appointment changing user; and transmitting, by the data system, the desired medical treatment date and the identification information of the medical treatment appointment changing user to the central management device.

According to the present disclosure, it is possible to provide a mobile medical treatment service system and a mobile medical treatment service method.

### BRIEF DESCRIPTION OF THE DRAWINGS

FIG. 1 illustrates a mobile medical treatment service system according to an embodiment.

FIG. 2 illustrates a drawing showing a data system and a central management device according to an embodiment.

FIG. 3 illustrates a flowchart of a method for determining a medical treatment schedule and a medical treatment subject in a medical treatment plan according to an embodiment.

FIG. 4 illustrates a flowchart of an appointment schedule changing method according to an embodiment.

### DETAILED DESCRIPTION OF THE EMBODIMENTS

The present disclosure relates to a mobile medical treatment service system that may determine an optimal position of the mobile medical treatment service system so that more users can use the mobile medical treatment service system.

The present disclosure will be described more fully hereinafter with reference to the accompanying drawings, in which embodiments of the disclosure are shown. As those skilled in the art would realize, the described embodiments may be modified in various different ways, all without departing from the spirit or scope of the present disclosure.

It is to be understood that when one constituent element is referred to as being “connected” or “coupled” to another constituent element, it may be connected or coupled directly to the other constituent element or may be connected or coupled to the other constituent element with a further constituent element intervening therebetween. In contrast, it should be understood that, when it is described that an element is “directly coupled” or “directly connected” to another element, no element is present between the element and the other element.

In the present application, it should be understood that the term “include”, “comprise”, “have”, or “configure” indicates that a feature, a number, a step, an operation, a constituent element, a part, or a combination thereof described in the specification is present, but does not exclude a possibility of presence or addition of one or more other features, numbers, steps, operations, constituent elements, parts, or combinations, in advance.

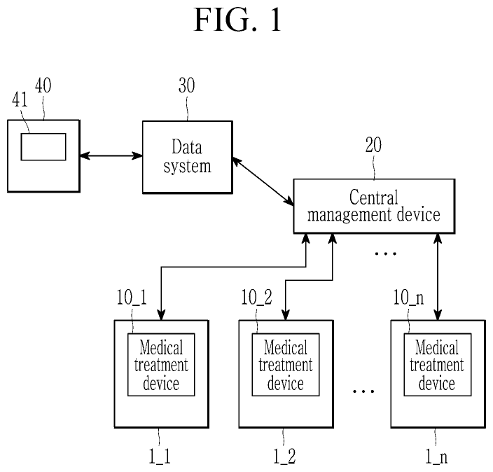

FIG. 1 illustrates a mobile medical treatment service system according to an embodiment.

Hereinafter, a medical treatment service provided through the mobile medical treatment service system is referred to as a “mobile medical treatment service”. A person who wants to use the mobile medical treatment service is called a service user, and an entity that manages the mobile medical treatment service is called a service provider.

A mobile medical treatment service system 1 includes at least one movable medical shelter 1_1 to 1_n. The movable medical shelter is hereinafter referred to as a mobile medical shelter. The mobile medical shelter may be implemented by installing necessary medical equipment in a modified bus. In the embodiment, the mobile medical treatment service system 1 is illustrated as including n mobile medical shelters (n is a natural number of greater than or equal to 1).

In the mobile medical shelter 1_1 to 1_n, a medical examination, a medical treatment, and the like are performed, and appropriate devices may be provided according to the performed medical treatment. Hereinafter, the device provided in the mobile medical shelter 1_1 to 1_n is defined as a medical treatment device. When medical treatment is performed in the mobile medical shelter 1_1 to 1_n, a doctor may be positioned in the mobile medical shelter 1_1 to 1_n, and when a medical examination is performed in the mobile medical shelter 1_1 to 1_n, a doctor or engineer specializing in examination may be positioned.

The mobile medical treatment service system 1 includes a plurality of medical treatment devices 10_1 to 10_n positioned in the mobile medical shelters 1_1 to 1_n, a central management device 20, and a data system 30. In order to receive the mobile medical treatment service, a mobile medical treatment service appointment person may install a mobile medical treatment application (an application program for receiving a mobile medical treatment service) 41 in a user terminal 40.

Information transmission and reception between the plurality of medical treatment devices 10_1 to 10_n, the central management device 20, the data system 30, and the user terminal 40 illustrated in FIG. 1 may be implemented by various communication methods through a wired/wireless network.

Each of the plurality of medical treatment devices 10_1 to 10_n may receive information on a condition of a service user from the central management device 20, and may display the condition of the service user. Medical staff who are positioned in the plurality of medical shelters 1_1 to 1_n may prepare actions for necessary treatment, examination, and the like according to the displayed condition of the service user. Each of the plurality of medical treatment devices 10_1 to 10_n includes various configurations necessary to perform actions such as the medical treatment and the examination to the service user for at least one specific medical subject. Each of the plurality of treatment devices 10_1 to 10_n may be fixed to a specific position, and may process information about a medical treatment result inputted by a doctor performing medical treatment to transmit it to an adjacent medical treatment device and the central management device 20. In addition, each of the plurality of medical treatment devices 10_1 to 10_n may display the received information about the service user to the outside so that the received information may be used for medical treatment. The information about the service user may include condition information of the service user.

The central management device 20 may derive information about the mobile medical treatment service appointment person from the data system 30 to determine a medical treatment plan including a medical treatment schedule, a medical treatment subject, and a medical treatment position at which the mobile medical treatment service system is located, for performing the mobile medical treatment.

The data system 30 may receive a medical treatment service appointment request and medical treatment appointment information through wired/wireless communication with the user terminal, and transmit appointment completion information to the user terminal that has transmitted the appointment request and the medical treatment appointment information in response to the appointment request.

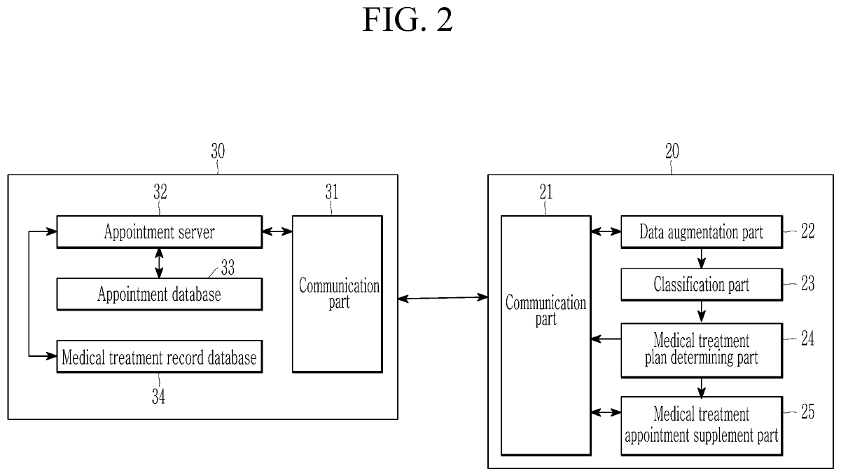

FIG. 2 illustrates a drawing showing a data system and a central management device according to an embodiment. The data system, central management device, and/or each of their components may each include at least one processor performing the various disclosed functions and may be implemented with software instructions executed on the at least one processor.

The data system 30 includes a communication part 31 providing wired/wireless communication, an appointment server 32, an appointment database 33, and a medical treatment record database 34. The appointment server 32 may receive an appointment request and medical treatment appointment information for the mobile medical treatment service through the communication part 31, may store the medical treatment appointment information as a response to the appointment request, and may additionally process an appointment schedule change by a request of the central management device 20.

The service provider inputs a medical treatment plan for a predetermined period for a specific area of a predetermined size through a user interface provided from the central management device 20. The user interface may be provided to the service provider terminal through the web. A method of implementing a specific user interface is not limited.

The mobile medical treatment service appointment person may input personal information, a desired medical treatment date, a residential area, and a symptom of the mobile medical treatment service appointment person through the mobile medical treatment application 41 installed in the user terminal. The personal information may include name, gender, age, phone number, and the like, and the personal information, the desired medical treatment date, the residential area, and the symptom may be comprehensively referred to as treatment appointment information.

The data system 30 receives a plurality of appointment requests and a plurality of pieces of medical treatment appointment information from the user terminals of the plurality of mobile medical treatment service appointment persons through the communication part 31. The appointment server 32 may store a plurality of pieces of the received medical treatment appointment information in the appointment database 33. The appointment database 33 may bundle and store the personal information, the desired medical treatment date, the residential area, and the symptom for each mobile medical treatment service appointment person. A storage basic unit in the appointment database 33 includes a plurality of data fields, and each of the plurality of data fields may store information (hereinafter, identification information) for identifying a mobile medical treatment service appointment person, personal information, a desired medical treatment date, a residential area, and a symptom, respectively.

The data system 30 further includes the medical treatment record database 34 for storing medical treatment information on a service user (hereinafter referred to as an existing service user) who has previously used a mobile medical treatment service. The medical information may include a symptom and a medical treatment detail during medical treatment, personal information about the existing service user, and information on a residential area. The storage basic unit in the medical treatment record database 34 may also include a plurality of data fields, and each of the plurality of data fields may store identification information, personal information, a symptom and medical details, and a residential area.

The central management device 20 includes a communication part 21, a data augmentation part 22, a classification part 23, a medical treatment plan determining part 24, and a medical treatment appointment supplement part 25.

The communication part 21 may transmit/receive information necessary to provide a mobile medical treatment service through a wired/wireless network. The information necessary for the central management device 20 to determine the medical treatment plan may be received through the communication part 21, and the determined medical treatment plan may be transmitted to a user terminal of the mobile medical treatment service appointment user and a user terminal of the preliminary service user through the communication part 21.

The data augmentation part 22 may read identification information, a desired treatment date, a residential area, and a symptom from each of the plurality of medical treatment appointment information from the data system 30.

The classification part 23 may generate a plurality of medical treatment appointment groups by classifying the plurality of mobile medical treatment appointments according to the desired medical treatment date, the residential area, and the symptom of each of the plurality of pieces of medical treatment appointment information read by the data augmentation part 22.

The medical treatment plan determining part 24 counts the number of the mobile medical treatment appointments included in each of the plurality of medical treatment appointment groups, may select a medical treatment appointment group with the highest number of the counted mobile medical treatment appointments among the plurality of medical treatment appointment groups as a medical treatment scheduled group, and may determine a medical treatment plan based on a desired medical treatment date, a symptom, and a residential area of the medical treatment scheduled group. The medical treatment plan determining part 24 may determine a medical treatment schedule based on the desired medical treatment date of the medical treatment scheduled group, may determine the symptom of the medical treatment scheduled group as a medical treatment subject, and may determine the residential area of the medical treatment scheduled group as the medical treatment target area. The medical treatment plan determining part 24 may machine-learn a relationship between an accumulated medical treatment position and a participation rate, and may determine a specific position in the medical treatment target area as a medical treatment position based on the machine-learned result. Specifically, the medical treatment plan determining part 24 may configure a participation rate predicting model for determining the medical treatment position, and may deep-learn the relationship between the accumulated medical treatment position and the participation rate. The participation rate refers to a ratio of users who have actual received mobile medical treatment services among all users who have made a medical treatment appointment for a confirmed medical treatment plan. For example, when 70 out of 100 appointment persons receive mobile medical treatment services, the participation rate may be 70%.

The medical treatment appointment supplement part 25 derives a medical treatment appointment group that does not match the medical treatment plan but has the same symptom among the plurality of medical treatment appointment groups, and may derive a phone number from personal information of a medical treatment appointment changing user, who is a mobile medical treatment service appointment person belonging to the derived medical treatment appointment group, and transmit the treatment plan to a telephone number of the medical treatment appointment changing user. The medical treatment appointment supplement part 25 may derive a phone number from the personal information of the preliminary service user stored in the data system 30, and transmit the medical treatment plan to the phone number of the preliminary service user. The preliminary service user may be an existing service user using a mobile medical service with the same symptom as the medical treatment plan.

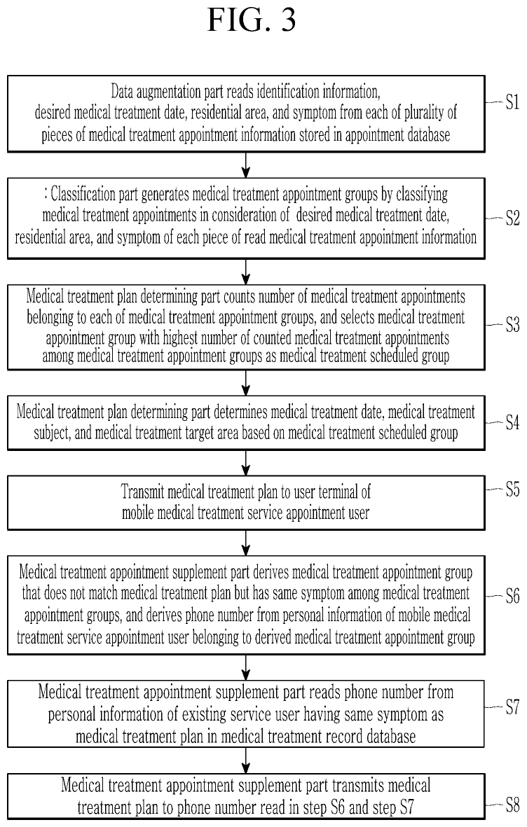

FIG. 3 illustrates a flowchart of a method for determining a medical treatment schedule and a medical treatment subject in a medical treatment plan according to an embodiment.

In the embodiment, medical treatment days are grouped into week units. The mobile medical treatment service appointment user may apply for medical treatment in a “desired month and week” through the mobile medical treatment application 41.

The data augmentation part 22 reads identification information, a desired medical treatment date, a residential area, and a symptom from each of the plurality of pieces of medical treatment appointment information stored in the appointment database 33 (S1).

The classification part 23 generates a plurality of medical treatment appointment groups by classifying the plurality of mobile medical treatment appointments in consideration of the desired medical treatment date, the residential area, and the symptom of each of a plurality of pieces of the read medical treatment appointment information. For example, the classification part 23 may receive the desired medical treatment date, the residential area, and the symptom read by the data augmentation part 22, may group the desired medical treatment date by week units, and may group medical appointments having similar symptoms and adjacent residential areas for each week into one group (S2). The classification part 23 may classify a plurality of mobile medical treatment appointments to generate a plurality of medical treatment appointment groups.

The medical treatment plan determining part 24 counts the number of mobile medical treatment appointments belonging to each of the plurality of medical treatment appointment groups, and selects a medical treatment appointment group with the highest number of the counted mobile medical treatment appointments among the plurality of medical treatment appointment groups as a medical treatment scheduled group (S3).

The medical treatment plan determining part 24 may determine a medical treatment date, a medical treatment subject, and a medical treatment target area based on the medical treatment scheduled group (S4).

For example, the central management device 20 generates a plurality of medical treatment appointment groups for each of the first week to the fourth week of July, based on symptoms and a residential area. Table 1 below summarizes the number of the plurality of medical treatment appointment groups for each week.

TABLE 1

| | | |
|---|---|---|
| First | Symptom ‘A’ & Area ‘a’ | Symptom ‘B’ & Area ‘b’ |
| week of | 20 | 15 |
| July | | |
| Second | Symptom ‘A’ & Area ‘c’ | Symptom ‘C’ & Area ‘b’ |
| week of | 12 | 9 |
| July | | |
| Third | Symptom ‘A’ & Area ‘a’ | Symptom ‘B’ & Area ‘b’ |
| week of | 11 | 13 |
| July | | |
| Fourth | Symptom ‘C’ & Area ‘a’ | Symptom ‘B’ & Area ‘a’ |
| week of | 8 | 17 |
| July | | |

The medical treatment plan determining part 24 may determine the first week of July, which has the highest count result, as a medical treatment schedule, the symptom ‘A’ with the largest number of applications for mobile medical treatment service as a medical treatment subject, and the residential area ‘a’ as a medical treatment target area. In the above example, it is described that the medical treatment dates are grouped by a week unit and only one of the plurality of medical treatment appointment groups is selected, but the present disclosure is not limited thereto. The medical treatment dates may be grouped into periods of a week or less, and two or more medical treatment appointment groups may be selected. The control unit may treat the symptom ‘A’ at a medical treatment position in the area ‘a’ in the first week, and may treat the symptom ‘B’ at a medical treatment position within the area ‘a’ in the fourth week.

The medical treatment plan determining part 24 may determine a feature position within the medical treatment target area as the medical treatment position by using deep learning. The medical treatment plan determining part 24 may learn the accumulated data on the medical treatment position and participation rate, and may specify a treatment position that is expected to have the highest participation rate among the residential areas of the medical treatment scheduled group based on the learned result (S4). The participation rate may be determined according to the number of service users who have actually received medical treatment compared to the number of mobile medical treatment appointments.

The medical treatment plan determining part 24 may transmit the determined medical treatment plan (medical treatment schedule, medical treatment kind, and medical treatment position) to the communication part 21, and the communication part 21 may transmit the medical treatment plan to the user terminal 40 of the mobile medical treatment service appointment user (S5).

The medical treatment appointment supplement part 25 may access the appointment database 33 through the communication part 21 to derive a medical treatment appointment group that does not match the medical treatment plan but has the same symptom among the plurality of medical treatment appointment groups, and may derive a phone number from the personal information of the mobile medical treatment service appointment user (hereinafter, the medical treatment appointment changing user) belonging to the derived medical treatment appointment group (S6). For example, in Table 1, the medical treatment appointment supplement part 25 may derive the medical treatment appointment groups for each of the symptom ‘A’ & the area ‘c’ of the second week of July and the symptom ‘A’ & the area ‘a’ of the third week of July. The medical treatment appointment supplement part 25 may derive phone numbers for 23 medical treatment appointment changing users belonging to the derived medical treatment appointment group.

In addition, the medical treatment appointment supplement part 25 may access the medical treatment record database 34 through the communication part 21 to read the phone number from the personal information of the existing service user having the same symptom as the medical treatment plan in the medical treatment record database 34 (S7).

The medical treatment appointment supplement part 25 may transmit the medical treatment plan to the phone number read in step S6 and step S7 (S8). This is to induce the medical treatment appointment changing user and the existing service user who has not appointed a mobile medical treatment service to be able to use the mobile medical treatment service according to the medical treatment plan. The medical treatment appointment changing user and the existing service user who has not appointed the mobile medical treatment service are referred to as preliminary service users.

The preliminary service user receiving the medical treatment plan may use the mobile medical treatment service according to the medical treatment plan.

Additionally, the central management device 20 may inquire about whether to change the appointment according to the medical treatment plan for the medical treatment appointment changing user, and may change the appointment schedule upon receiving a response to the inquiry.

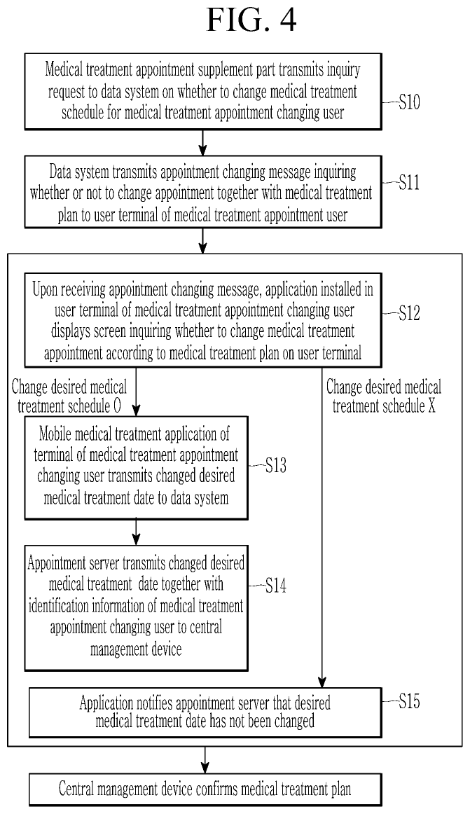

FIG. 4 illustrates a flowchart of an appointment schedule changing method according to an embodiment.

The medical treatment appointment supplement part 25 transmits an inquiry request on whether to change the medical treatment schedule for the medical treatment appointment changing user through the communication part 21 to the data system 30 (S10). In this case, the medical treatment appointment supplement part 25 may transmit the identification number of the medical treatment appointment changing user to the data system 30 together therewith.

The data system 30 may transmit an appointment changing message inquiring whether or not to change the appointment together with the medical treatment plan to the user terminal of the medical treatment appointment changing user (S11). For example, the appointment server 32 may read the phone number of the medical treatment appointment changing user from the appointment database 33 based on the identification information received from the medical treatment appointment supplement part 25, and may transmit the appointment changing message inquiring whether to change the medical treatment appointment to the corresponding phone number.

Upon receiving the appointment changing message, the mobile medical treatment application 41 installed in the user terminal of the medical treatment appointment changing user may display a screen inquiring whether to change the medical treatment appointment according to the medical treatment plan on the user terminal (S12).

The medical treatment appointment changing user may change the desired medical treatment date according to the medical treatment plan in response to the inquiry. The mobile medical treatment application 41 of the user terminal of the medical treatment appointment changing user transmits the changed desired medical treatment date to the data system 30 (S13).

The appointment server 32 of the data system 30 receives the changed desired medical treatment date, and transmits the changed desired medical treatment date together with the identification information of the medical treatment appointment changing user to the central management device 20 (S14).

When the medical treatment appointment changing user does not change the desired medical treatment date through the mobile medical treatment application 41, the mobile medical treatment application 41 may notify the appointment server 32 that the desired medical treatment date has not been changed (S15).

In step S12 to step S15, the desired medical treatment date in the appointment server 32 is changed or maintained through the mobile medical treatment application 41 of all user terminals of additional users, the appointment server 32 may transmit the result to the central management device 20, and the central management device 20 may confirm the medical treatment plan according to the received result (S16).

By inducing change of the medical treatment date to the medical treatment appointment changing user, it is possible to more accurately predict the number of users who will use the mobile medical treatment service. In addition, the medical treatment appointment changing user, and the existing service user who has not changed their medical treatment schedule, may still receive the mobile medical treatment services when they visit the mobile medical treatment service system according to their medical plans.

While this disclosure has been described in connection with what is presently considered to be practical embodiments, it is to be understood that the disclosure is not limited to the disclosed embodiments, but, on the contrary, is intended to cover various modifications and equivalent arrangements included within the spirit and scope of the appended claims.

What is claimed is:

1. A mobile medical treatment service system, comprising:
a data system configured to receive a plurality of mobile medical treatment appointment requests and a plurality of pieces of medical treatment appointment information from a plurality of user terminals, and to store the plurality of pieces of medical treatment appointment information; and
a central management device configured to derive the plurality of pieces of medical treatment appointment information from the data system,
to classify a plurality of mobile medical treatment appointments into a plurality of groups based on a desired medical treatment date among the derived plurality of pieces of medical treatment appointment information, and to determine a medical treatment schedule according to a number of appointments belonging to the plurality of groups,
to calculate a medical treatment target area based on residential areas of a plurality of users among the derived plurality of pieces of medical treatment appointment information, and
to determine a medical treatment position within the medical treatment target area by machine-learning a relationship between an accumulated medical treatment position and a participation rate,
wherein the participation rate is a ratio of users that have actually received a mobile medical treatment service among the plurality of users.

2. The mobile medical treatment service system of claim 1, wherein the central management device includes:
a data augmentation part configured to read identification information, a desired medical treatment date, a residential area, and a symptom from each of the plurality of pieces of medical treatment appointment information from the data system;
a classification part configured to generate a plurality of medical treatment appointment groups by classifying the plurality of mobile medical treatment appointments according to the desired medical treatment date, the residential area, and the symptom of each of the read plurality of pieces of medical treatment appointment information; and
a medical treatment plan determining part configured to count a number of the mobile medical treatment appointments belonging to each of the plurality of medical treatment appointment groups, to select a medical treatment appointment group with the highest number of the counted mobile medical treatment appointments among the plurality of medical treatment appointment groups as a medical treatment scheduled group, and to determine a medical treatment plan based on a desired medical treatment date, a symptom, and a residential area of the medical treatment scheduled group.

3. The mobile medical treatment service system of claim 2, wherein
the medical treatment plan determining part determines a medical treatment schedule based on the desired medical treatment date of the medical treatment scheduled group, determines the symptom of the medical treatment scheduled group as a treatment subject, and determines the residential area of the medical treatment scheduled group as the medical treatment target area.

4. The mobile medical treatment service system of claim 3, wherein
the medical treatment plan determining part learns the relationship between the accumulated medical treatment position and the participation rate through deep learning, and determines a specific position within the medical treatment target area as the medical treatment position.

5. The mobile medical treatment service system of claim 2, wherein
the central management device includes a medical treatment appointment supplement part configured to derive a medical treatment appointment group that does not match the medical treatment plan but has the same symptom among the plurality of medical treatment appointment groups, to derive a phone number from personal information of a medical treatment appointment changing user who is a mobile medical treatment service appointment user belonging to the derived medical treatment appointment group, and to transmit the medical treatment plan to the phone number of the medical treatment appointment changing user.

6. The mobile medical treatment service system of claim 5, wherein
the medical treatment appointment supplement part derives a phone number from personal information of a preliminary service user stored in the data system to transmit the medical treatment plan to the phone number of the preliminary service user, and
the preliminary service user is an existing service user using a mobile medical treatment service with the same symptom as the medical treatment plan.

7. The mobile medical treatment service system of claim 2, wherein
the central management device further includes:
a medical treatment appointment supplement part configured to derive a medical treatment appointment group that does not match the medical treatment plan but has the same symptom among the plurality of medical treatment appointment groups, and to derive an identification number of a medical treatment appointment changing user who is a mobile medical treatment service appointment user belonging to the derived medical treatment appointment group to transmit it to the data system.

8. The mobile medical treatment service system of claim 7, wherein
the data system transmits the medical treatment plan to a phone number corresponding to the derived identification number, receives a changed desired medical treatment date from a user terminal of the medical treatment appointment changing user, and transmits the desired medical treatment date and identification information of the medical treatment appointment changing user to the central management device.

9. The mobile medical treatment service system of claim 1, wherein
the data system includes an appointment server configured to store at least one of the plurality of pieces of medical treatment appointment information in response to at least one of the plurality of mobile medical treatment appointment requests for the mobile medical treatment service and additionally processes an appointment schedule change in response to a request from the central management device.

10. The mobile medical treatment service system of claim 9, wherein
the appointment server includes:
an appointment database configured to store personal information, a desired treatment date, a residential area, and a symptom for each mobile medical treatment service appointment user; and
a medical treatment record database configured to store medical treatment information about a service user who has previously used a mobile medical treatment service.

11. A mobile medical treatment service method, comprising:
receiving, by a data system, a plurality of mobile medical treatment appointment requests and a plurality of pieces of medical treatment appointment information from a plurality of user terminals, and storing the plurality of pieces of medical treatment appointment information;
deriving, by a central management device, a plurality of pieces of medical treatment appointment information from the data system;
classifying, by the central management device, a plurality of mobile medical treatment appointments into a plurality of groups based on a desired medical treatment date among the derived plurality of pieces of medical treatment appointment information;
determining, by the central management device, a medical treatment schedule according to a number of appointments belonging to the plurality of groups;
calculating, by the central management device, a medical treatment target area based on residential areas of a plurality of users among the derived plurality of pieces of medical treatment appointment information;
learning, by the central management device, a relationship between an accumulated medical treatment position and a participation rate through deep learning; and
determining, by the central management device, a medical treatment position in the medical treatment target area based on a learned result, wherein the participation rate is a ratio of users that have actually received a mobile medical treatment service among the plurality of users.

12. The mobile medical treatment service method of claim 11, wherein
the classifying of the plurality of mobile medical treatment appointments into the plurality of groups includes:
reading, by the central management device, identification information, a desired medical treatment date, a residential area, and a symptom from each of the plurality of pieces of medical treatment appointment information from the data system; and
generating, by the central management device, a plurality of medical treatment appointment groups by classifying the plurality of mobile medical treatment appointments in consideration of the desired medical treatment date, the residential area, and the symptom of each of the read plurality of pieces of medical treatment appointment information.

13. The mobile medical treatment service method of claim 12, wherein
the determining includes:
counting, by the central management device, a number of the mobile medical treatment appointments belonging to each of the plurality of medical treatment appointment groups;
selecting, by the central management device, a medical treatment appointment group having the highest number of the mobile medical treatment appointments counted among the plurality of medical treatment appointment groups as a medical treatment scheduled group;
determining, by the central management device, the medical treatment schedule based on a desired medical treatment date of the medical treatment scheduled group;
determining, by the central management device, a symptom of the medical treatment scheduled group as a medical treatment subject; and
determining, by the central management device, a residential area of the medical treatment scheduled group as the medical treatment target area.

14. The mobile medical treatment service method of claim 13, wherein
the determining of the medical treatment position includes determining, by the central management device, a specific position in the medical treatment target area as the medical treatment position based on the learned result.

15. The mobile medical treatment service method of claim 12, further comprising:
deriving, by the central management device, a medical treatment appointment group that does not match a medical treatment plan but has the same symptom among the plurality of medical treatment appointment groups;
deriving, by the central management device, a phone number from personal information of a medical treatment appointment changing user who is a mobile medical treatment service appointment user belonging to the derived medical treatment appointment group; and
transmitting, by the central management device, the medical treatment plan to the phone number of the medical treatment appointment changing user.

16. The mobile medical treatment service method of claim 15, further comprising
deriving, by the central management device, a phone number from personal information of a preliminary service user stored in the data system; and
transmitting, by the central management device, the medical treatment plan to the phone number of the preliminary service user,
wherein the preliminary service user is an existing service user using a mobile medical treatment service with the same symptom as the medical treatment plan.

17. The mobile medical treatment service method of claim 12, further comprising:
deriving, by the central management device, a medical treatment appointment group that does not match a medical treatment plan but has the same symptom among the plurality of medical treatment appointment groups;
deriving, by the central management device, an identification number of a medical treatment appointment changing user who is a mobile medical treatment service appointment user belonging to the derived medical treatment appointment group; and
transmitting, by the central management device, the derived identification number to the data system.

18. The mobile medical treatment service method of claim 17, further comprising
transmitting, by the data system, the medical treatment plan to a phone number corresponding to the derived identification number;
receiving, by the data system, a changed desired medical treatment date from a user terminal of the medical treatment appointment changing user; and
transmitting, by the data system, the desired medical treatment date and the identification information of the medical treatment appointment changing user to the central management device.
